(19) United States
(12) Patent Application Publication
(10) Pub. No.: US 2026/0007332 A1
(43) Pub. Date: Jan. 8, 2026

(54) System For Using Headphones Around Neck to Estimate Personal Attenuation Ratings of Hearing Protection Devices and Associated Methods

(71) Applicant: The Government of the United States, as Represented by the Director of the Defense Health Agency, Fort Detrick, MD (US)

(72) Inventors: Douglas S. BRUNGART, Rockville, MD (US); Devon M. KULINSKI, Attleboro, MA (US); Julianna R. VOELKER, Washington, DC (US)

(21) Appl. No.: 19/256,635

(22) Filed: Jul. 1, 2025

Related U.S. Application Data
(60) Provisional application No. 63/667,109, filed on Jul. 2, 2024.

(51) Int. Cl. A61B 5/12; A61B 5/00; A61F 11/08; A61F 11/14; G06F 3/16

(52) U.S. Cl. CPC A61B 5/123; A61B 5/0002; A61B 5/6803; A61B 5/7435; A61B 5/7475; A61F 11/08; A61F 11/14; G06F 3/165; A61B 2560/0242

(57) ABSTRACT

A neck-based personal attenuation rating (PAR) system is used for determining a personal attenuation rating (PAR) of a hearing protection device on a listener. Headphones with an audiometer are placed around ears of the listener, and the audiometer is operated to measure an on-ear hearing threshold of the listener without the hearing protection device. The headphones with the audiometer may then be placed around a neck of the listener, and the audiometer is operated to measure an on-neck hearing threshold of the listener with the hearing protection device. A neck-to-ear transfer function is applied to a difference in the measured on-ear and on-neck hearing thresholds to determine a measured personal attenuation rating of the hearing protection device. A determination is made on if an ambient noise level in an area surrounding the listener compromised the measured personal attenuation rating. This determines if the measured PAR is reliable.

### RELATED APPLICATION

The present application claims the benefit of U.S. Provisional Patent Application Ser. No. 63/667,109 filed Jul. 2, 2024, all of which is fully incorporated by reference.

### STATEMENT OF GOVERNMENT INTEREST

The invention(s) described herein may be manufactured, used, and/or licensed by or for the Government of the United States of America without payment by the Government of any royalties thereon.

### TECHNICAL FIELD

The present disclosure relates to hearing protection devices, and, more particularly, to a system for estimating personal attenuation ratings of hearing protection devices and associated methods.

### BACKGROUND

Hearing protection devices (HPDs) play a vitally important role in preventing hearing loss for individuals who are exposed to high levels of noise. However, the amount of protection that a particular HPD provides depends on a large number of factors. These factors include how well the HPD is fitted to the size and shape of the user's head and ears, how well trained the user is in properly fitting the HPD, how careful the user is in properly inserting or donning the HPD, and whether there has been any deterioration in the function of the device due to age or wear.

The U.S. Environmental Protection Agency (EPA) has established standards for estimating the amount of hearing protection a device will provide when properly sized and inserted into the ears by a well-trained individual (1979). This information is captured by the Noise Reduction Rating (NRR), which is designed to estimate the minimum amount of attenuation a well-fit HPD will provide for 95% of the population. However, a number of studies have shown that the NRR is not an accurate estimate of the amount of attenuation users obtain from HPDs in real-world environments.

In 1977 and 1981, the National Institute for Occupational Health and Safety (NIOSH) evaluated the attenuation provided by earplug HPDs for 420 workers at 15 industrial plants and found that 50% of workers were obtaining attenuation values that were less than one half the NRR (Lempert and Edwards, 1983). This led to new OSHA guidelines requiring hearing conservation professionals to “de-rate” the NRR value when calculating the amount of attenuation a particular HPD will provide in a noise environment (OSHA, 2013).

Currently, the National Institute for Occupational Safety and Health (NIOSH) recommends subtracting 25% from the manufacturer's published NRR of earmuffs, 50% from the manufacturer's published NRR of formable earplugs, and 70% from the manufacturer's published NRR for all other earplugs (Health et al., 1998). De-rating provides a much more realistic estimate of the attenuation an HPD is likely to provide in real-world use than the published NRR, but the de-rated NRR is still very flawed as a predictor of the attenuation a particular individual will obtain from a particular HPD.

Many users fail to obtain even the de-rated NRR of a hearing protection device if it does not fit properly or they are not trained to use it properly. For example, Federman et al. (2021) evaluated the actual attenuation provided by foam earplugs inserted by 119 untrained Marine Corps recruits and found that none were able to achieve the published NRR of 33 dB. Roughly 33% obtained attenuation values lower than the 16.5 dB value that would be expected with a 50% de-rated NRR, and that more than 10% obtained attenuation values less than the 10 dB value that would be expected with a 70% re-rated NRR.

Similar numbers were reported by Murphy et al. (2022). There are also cases where individual users are exposed to noise environments that are so loud that it is impossible to achieve a safe exposure level with the de-rated attenuation of any commercially available HPD. In these environments, it may be necessary to verify that the HPD is providing enough attenuation to protect from the noise environment, rather than to simply rely on the de-rated NRR.

In order to address these issues, hearing conservation professionals have developed devices known as Field Attenuation Estimation Systems (FAES) that are capable of measuring the amount of protection a particular HPD is providing to a particular listener at the time of the test. There are two basic kinds of FAES systems: electroacoustic systems and psychoacoustic systems. Electroacoustic systems use physical measurements to estimate the attenuation provided by the device. Psychoacoustic systems use behavioral testing to estimate the amount of attenuation provided by the device.

Electroacoustic FAESs are conceptually very simple. They use a Microphone-in-Real-Ear (MIRE) method where a microphone is inserted inside the ear underneath the HPD and the sound level at that microphone is compared to the noise level measured with a second microphone located outside of the HPD. The difference between the two noise levels can be used to calculate the attenuation provided by the HPD in each frequency band. These individual frequency-dependent attenuation values can be used to calculate a single number (the Personal Attenuation Rating or PAR) that approximates the number of a-weighted decibels of attenuation the HPD would provide if the user was in a broadband noise environment.

The problem with this method is that it is not trivial to find a way to insert the microphone under the HPD without interfering with the amount of attenuation it provides. The most commonly used electroacoustic FAES addresses this problem by replacing the HPD with a “surrogate” hearing protector that has a probe tube passing through the earplug. However, it can only be used on a limited number of hearing protectors, and it is not possible to examine the effects that use and wear-and-tear might have on the attenuation provided the specific HPD that is currently in use.

Psychoacoustic FAES systems use human behavioral testing to estimate the attenuation provided by an HPD. The most common method for this kind of test is the Real-Ear Attenuation at Threshold (REAT) method, where the absolute hearing threshold is measured with the HPD inserted and with the open ear. The difference in thresholds between the two conditions is used to estimate the attenuation of the device.

The ANSI standard for measuring the NRR is based on the REAT, and the REAT is considered the gold standard for measuring the attenuation provided by HPDs (ANSI 2020). However, the ANSI standard for conducting REAT testing has strict requirements for a double-walled, sound-isolated reverberation room capable of producing a perfectly diffuse sound field. This is something that is only available in a small number of laboratories.

FAES systems based on the REAT standard replace the diffuse free-field sound field with a pair of oversized headphones that can be placed over most earplug HPDs. There are also FAES systems that use oversized headphones to present sounds to one occluded ear and one unoccluded ear, and use a loudness balancing method to approximate the attenuation provided by the earplug. Headphone based FAES systems work well for testing earplug hearing protectors, but they are not suitable for making PAR measurements wearing earmuffs rather than earplugs.

One limitation of all currently available FAES systems is that they are unable to accurately measure the amount of attenuation provided by double hearing protection, where a listener wears earplugs and earmuffs at the same time.

Psychoacoustic FAES systems cannot test double protection because there is no way to place the headphones they use to generate sounds over earmuffs. Electroacoustic systems cannot test double protection because they do not provide a way to place one microphone under the earplug and the second microphone outside the earmuff. Even if this were possible, MIRE estimates of double HPD attenuation are generally not reliable because the sound path to the cochlea is dominated by flanking pathways (bone and tissue conduction) that are capable of causing hearing damage even when 100% of the sound entering through the ear canal is eliminated (Berger et al. 2003).

### SUMMARY

A method for determining a personal attenuation rating of a hearing protection device on a listener using a neck-based PAR system includes placing headphones with an audiometer or other calibrated noise source around ears of the listener, and operating the audiometer to measure an on-ear hearing threshold of the listener without the hearing protection device. The headphones from the audiometer are then placed around a neck of the listener, and the audiometer is operated to measure an on-neck hearing threshold of the listener with the hearing protection device. A neck-to-ear transfer function is applied to a difference in the measured on-ear and on-neck hearing thresholds to determine a measured personal attenuation rating of the hearing protection device. A determination is made if an ambient noise level in an area surrounding the listener compromised the measured personal attenuation rating. If the ambient noise level in the area did not compromise the measured personal attenuation rating, then the measured personal attenuation rating is reliable. Alternatively, if the ambient noise level in the area did compromise the measured personal attenuation rating, then the measured personal attenuation rating is not reliable.

The hearing protection device worn by the listener may comprise ear muffs, where the size of the headphones with the audiometer will not fit over the earmuffs. Alternatively, the hearing protection device worn by the listener may comprise both ear plugs and ear muffs, where the size of the headphones with the audiometer will not fit over both the ear plugs and the earmuffs.

The method may further include determining the neck-to ear transfer function by placing headphones with an audiometer around the ears of an acoustic manikin, operating the audiometer to generate a test signal, with the test signal being used to determine on-ear frequency responses in the ears of the acoustic manikin. The headphones with the audiometer are then placed around the neck of the acoustic manikin, while the audiometer is operated to generate the test signal, with the test signal being used to determine on-neck frequency responses in the ears of the acoustic manikin. A difference between the frequency spectrum of the on-ear measurements and the frequency spectrum of the on-neck measurements is used to determine the neck-to-ear transfer function.

The listener operates the audiometer. The audiometer may be configured to generate tones and is coupled to a tablet computing device that includes a touch screen, and wherein the listener operates the audiometer via the touch screen. The audiometer and the tablet computing device may be wirelessly coupled together.

The touch screen may be configured to provide a response button, and wherein the listener operates the audiometer holding down the response button when the tone is heard, and releasing the response button when the tone is no longer heard.

The headphones include spaced apart earcups, with the earcups being angled away from the listener when the headphones are placed around the neck of the listener.

Another aspect is directed to a neck-based personal attenuation rating (PAR) system comprising headphones with an audiometer or other calibrated noise source, a computing device, and an ambient noise level detector. The computing device includes a touchscreen with a response button to be operated by the listener to determine an on ear threshold while the headphones with the audiometer are placed over ears of a listener not wearing a hearing protection device, and an on neck threshold while the headphones with the audiometer are placed around a neck of the listener while wearing the hearing protection device. A memory in the computing device is configured to store the on ear threshold and the on neck threshold. A processor is coupled to the touchscreen and to the memory in the computing device, and is configured to apply a transfer function to a difference between the on ear threshold and the on neck threshold to determine a personal attenuation rating of the hearing protection device. The ambient noise level detector is configured to determine if an ambient noise level in an area surrounding the listener compromised the determined personal attenuation rating. The ambient noise level detector may be carried by the computing device.

### BRIEF DESCRIPTION OF THE DRAWINGS

FIG. 1 is a block diagram of a neck-based personal attenuation rating (PAR) system in which various aspects of the disclosure may be implemented.

FIG. 2 is a flowchart for determining the personal attenuation rating of a hearing protection device on a listener using the neck-based PAR system illustrated in FIG. 1.

FIG. 3 is a flowchart for determining a neck-to-ear transfer function used in determining the personal attenuation rating for the hearing protection device illustrated in FIG. 1.

FIG. 4 is a picture of a standard configuration for fitting the WAHTS headphones around the neck of the KEMAR manikin.

FIG. 5 is a graph of neck-to-ear transfer functions for the KEMAR manikin for six repetitions of standard fitting of the device on the neck.

FIG. 6B are pictures of the different positions of the WAHTS headphones around the neck of the KEMAR manikin.

FIG. 6A is a graph of neck-to-ear transfer functions for the KEMAR manikin with seven non-standard fittings of the device.

FIG. 7 is a graph of neck-to-ear transfer functions for seven human listeners with the WAHTS headphones placed in a standard configuration.

FIG. 8 is a plot comparing on-ear hearing thresholds measured with ⅓rd octave noise bands using modified Bekesy tracking method to audiometric pure tone thresholds measured as part of an annual hearing test.

FIG. 9 is a plot of raw hearing thresholds measured with ⅓rd octave noise bands using modified Bekesy tracking method for each of the five conditions of the experiment.

FIG. 10 is a plot of raw estimates of attenuation for each hearing protection conditions at each frequency, obtained by subtracting the on-neck threshold from the on-ear threshold.

FIGS. 11A-11C are plots on comparing raw (unadjusted) attenuations obtained by subtracting on-ear threshold from on-neck threshold at each frequency to frequency-by-frequency PAR84 values provided for the same fit with the EARFit system.

FIG. 12 is a plot of estimated on-neck PAR84 as a function of PAR84 measured with the EarFit system for 126 data points in the original validation study.

FIG. 13 is a plot of estimated on-neck PAR84 as a function of PAR84 measured with the EarFit system for 208 data points from the original validation study and a follow-on study that used occluded and unoccluded thresholds on the WAHTS headphones to estimate the PAR value.

FIGS. 14A-14C are plots of On-Neck PAR as a function of Measured PAR for all trials tests (FIG. 14A), just those trials where the test administrator reported that they did not hear at least one of the Bekesy tracks (FIG. 14B), and just those trials where the test administrator said they did hear all three Bekesy tracks (FIG. 14C).

### DETAILED DESCRIPTION

The present description is made with reference to the accompanying drawings, in which exemplary embodiments are shown. However, many different embodiments may be used, and thus the description should not be construed as limited to the particular embodiments set forth herein. Rather, these embodiments are provided so that this disclosure will be thorough and complete. Like numbers refer to like elements throughout.

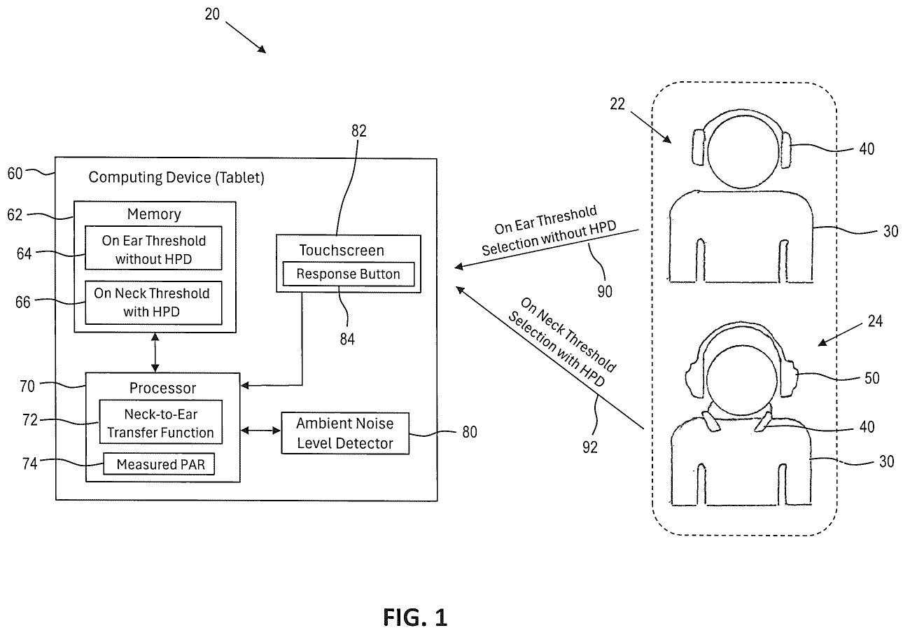

As will be discussed in detail below, a field attenuation estimation system (FAES) 20 is configured to measure how much protection a particular hearing protection device (HPD) 50 is providing to a particular listener 30 based on a neck-based sound source 40, as shown in FIG. 1. This system 20 will also be referred to as a neck-based personal attenuation rating (PAR) system.

The neck-based personal attenuation rating system 20 includes a computing device 60 and a sound source 40. The computing device 60 may be configured as a portable, handheld device, such as a tablet, that is to be carried by the listener 30. The sound source 40 is an audiometer configured as a pair of headphones commonly used in hearing tests, such as a WAHTS headphones or headset. The audiometer may be configured to include any system that uses a calibrated audio signal varying in level to measure thresholds. The WAHTS headphones may also be referred to as a wireless automated hearing test system.

The sound source 40 is configured to wirelessly connect to the computing device 60. The sound source 40 is positioned around the ears of the listener 30 when the listener 30 is not wearing a hearing protection device 50, as indicated by reference 22. This allows the listener 30 to operate a response button 84 on a touchscreen 82 of the computing device 60 to establish an on ear threshold selection 90. The on ear threshold selection 90 provides an on ear threshold without HPD 64 to be stored in a memory 62 within the computing device 60.

The listener 30 holds down the response button 84 when the tone is heard, and releases the response button 84 when the tone is no longer heard. When the response button 84 is pressed, the intensity level of the frequency tested will automatically be reduced. When the response button 84 is released, the intensity level will automatically increase. This hearing threshold testing may be referred to as the Bekesy audiometry test procedure method.

The sound source 40 is next positioned around the neck of the listener 30 while the listener 30 is wearing a hearing protection device 50, as indicated by reference 24. This allows the listener 30 to operate the response button 84 on the touchscreen 82 of the computing device 60 to establish an on neck threshold selection 92. The on neck threshold selection 92 provides an on neck threshold with HPD 66 to be stored in the memory 62 within the computing device 60.

The order of positioning the sound source 40 around the ears of the listener 30 when the listener 30 is not wearing a hearing protection device 50 (as indicated by reference 22), and positioning the sound source 40 around the neck of the listener 30 while the listener 30 is wearing a hearing protection device 50 (as indicated by reference 24) may be performed in a reverse order.

The computing device 60 includes a processor 70 coupled to the touchscreen 82 and to the memory 62. As will be discussed in greater detail below, the processor 70 is configured to determine a measured personal attenuation rating (PAR) 74 of the hearing protection device 50 as worn by the listener 30. The processor 70 applies a neck-to-ear transfer function 72 to a difference in the measured on ear threshold without HPD 64 and the on neck threshold with HPD 66. The neck-to-ear transfer function is to compensate for a difference in the level of the audible signals that reaches the listener 30 when the sound source 40 is worn around the neck (as indicated by reference 24) and when worn over the ears (as indicated by reference 22).

The neck-based personal attenuation rating system 20 further includes use of an ambient noise level detector 80. In one embodiment, the ambient noise level detector 80 is carried by the computing device 60. In this case, the ambient noise level detector 80 is coupled to the processor 70, and allows the processor 70 to determine if an ambient noise level in the area surrounding the listener 30 distorts the measured personal attenuation rating 74. If the ambient noise level is low, then the measured personal attenuation rating 74 of the hearing protection device 50 being tested is to be treated as reliable. In an alternate embodiment, the ambient noise level detector 80 may be separate or external the computing device 60 when measuring the ambient noise level in the area surrounding the listener 30.

Alternatively, or in addition to the noise level detector 80, a test administrator in the area is able to verify that the ambient noise level did not compromise the determined personal attenuation rating in response to hearing an audible test signal from the audiometer at a lowest level tested when measuring the on-neck hearing threshold.

Currently, the above-described sound source 40 is typically placed over the ears of the listener 30 to measure the personal attenuation rating of earplugs only. However, this approach cannot be used to measure the personal attenuation rating provided to the listener 30 while wearing earmuffs 50, or when wearing double hearing protection, such as earplugs and earmuffs 50. This is due to the sound source 40 not being sized to cover the earmuffs 50.

The neck-based PAR system 20 advantageously allows a personal attenuation rating to be determined for most any hearing protection device(s) being used by the listener. The neck-based PAR system 20 also allows for assessment of hearing protection devices that are worn in conjunction with helmets, eyeglasses, or other personal protective equipment (PPE).

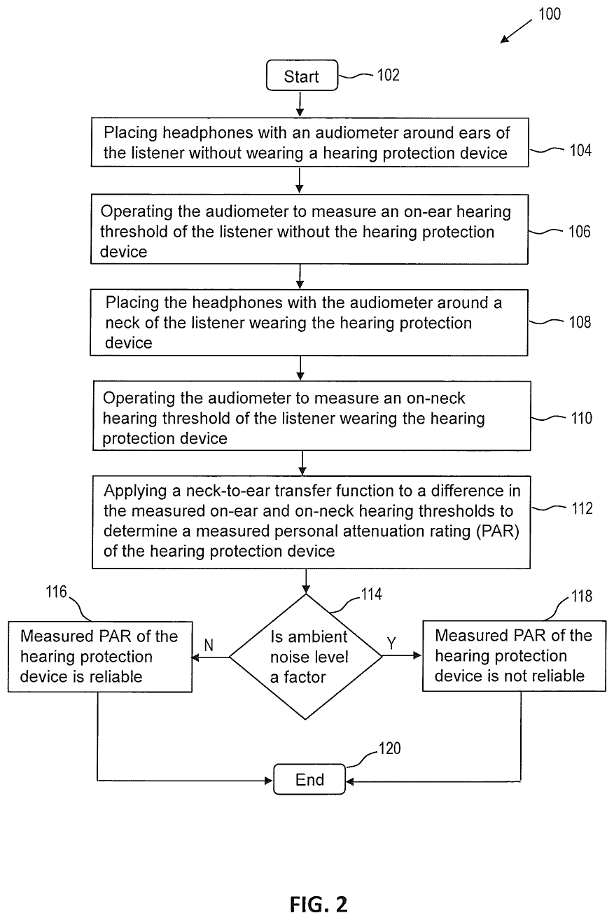

Referring now to the flowchart 100 in FIG. 2, a method for determining the personal attenuation rating (PAR) of a hearing protection device 50 on a listener 30 using the neck-based PAR system 20 will be discussed. The above-described sound source 40 will also be referred to as headphones 40 with an audiometer. From the start (Block 102), the headphones 40 with the audiometer are placed around the ears of the listener 30 at Block 104. The audiometer is operated at Block 106 via the tablet computing device 60 to measure a hearing threshold of the listener 30 without the hearing protection device. This measurement may be referred to as an on-ear threshold 64 or an unoccluded threshold.

The headphones 40 with the audiometer are then placed around the neck of the listener 30 at Block 108 while the listener is wearing the hearing protection device 50. The audiometer is operated at Block 110 via the tablet to measure a hearing threshold of the listener with the hearing protection device. This measurement may be referred to as an on-neck threshold 66 or an occluded threshold.

A neck-to-ear transfer function 72 is applied to a difference in the measured on-ear threshold 64 and the on-neck threshold 66 at Block 112 to determine a measured personal attenuation rating 74 of the hearing protection device 50. The neck-to-ear transfer function 72 is to compensate for a difference in the level of the signal that reaches the ears of the listener 30 from the headphones 40 with the audiometer worn around the neck, and the level of the signal that reaches the ears of the listener 30 when the headphones 40 with the audiometer are worn over the head.

The neck-to-ear transfer function 72 is calculated by taking a difference between the frequency spectrum of on-ear measurements and the frequency spectrum of the on-neck measurements, as will be discussed in greater detail below. Linear regression may be used to establish an empirical relationship between the different frequency spectrums of the on-ear measurements and the on-neck measurements.

A determination is made at Block 114 on if an ambient noise level in the area surrounding the listener 30 compromised or distorted the measured personal attenuation rating 74. Ambient noise can mask the test signal and artificially increase the measured thresholds. When this happens in the unoccluded condition, this will result in an artificial decrease in the estimated PAR value. When it happens in the occluded condition, it will result in an artificial increase in the PAR value.

If the ambient noise level is low, then at Block 116 the measured personal attenuation rating 74 of the hearing protection device 50 being tested is to be treated as reliable. If the ambient noise level is high and compromises the measurements, then at Block 118 the measured personal attenuation rating 74 of the hearing protection device 50 being tested is to be treated as not reliable. The method ends at Block 120.

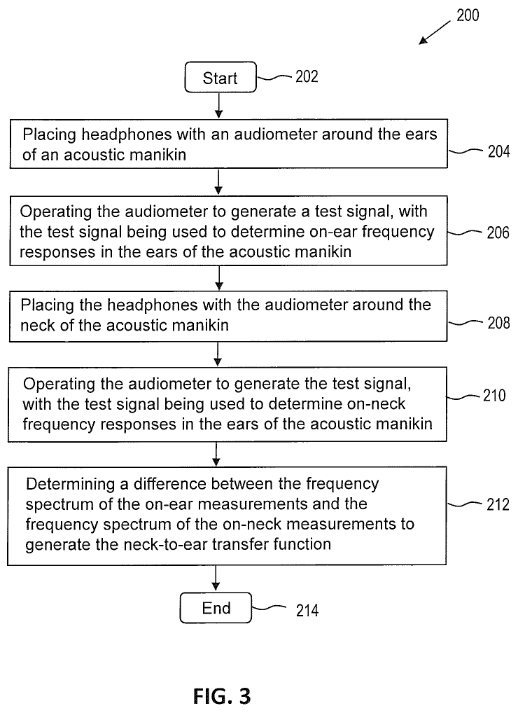

Referring now to the flowchart 200 in FIG. 3, a method for determining the neck-to-ear transfer function for the neck-based PAR system will be discussed. From the start (Block 202), the method includes placing headphones with an audiometer around the ears of an acoustic manikin at Block 204. The audiometer is operated at Block 206 to generate a test signal, with the test signal being used to determine on-ear frequency responses in the ears of the acoustic manikin.

The headphones with the audiometer are placed around the neck of the acoustic manikin at Block 208. The audiometer is operated at Block 210 to generate the test signal, with the test signal being used to determine on-neck frequency responses in the ears of the acoustic manikin. A difference between the frequency spectrum of the on-ear measurements and the frequency spectrum of the on-neck measurements is determined at Block 212 to determine the neck-to-ear transfer function. The method ends at Block 214.

As will be discussed in greater detail below, validation of the neck-to-ear transfer function may be performed by placing the headphones around the neck of the acoustic manikin for a variety of different fits. The headphones include a pair of earcups, and each fit is based on how the earcups are angled with respect to one another. The standardized configuration to be used when placing the headphones on the neck has earcups angled slightly away from the listener. For each fit of the headphones, a difference between the frequency spectrum of the on-ear measurements and the frequency spectrum of the on-neck measurements is determined to determine the neck-to-ear transfer function. Validation of the neck-to-ear transfer function may also be performed using humans instead of an acoustic manikin.

The standard deviation across the different fittings of the standardized configuration was never more than 4 dB, and at most frequencies it was less than 3 dB. The standard deviation across the non-standard configurations was less than 5 dB across all frequencies tested. This indicates that the neck-to-ear transfer function is very reliable for repeated placements of the headphones if reasonable care is taken to ensure the headphones are placed on the neck in the standardized configuration.

### Description and Operation

As noted above, the neck-based PAR system is a novel FAES system that uses a pair of headphones worn around the neck, rather than over the ears, as the sound source for measuring the PAR of an HPD. This around-the-neck placement makes it possible to make PAR estimates in the field for individuals wearing double hearing protection, as well as for those wearing earplugs alone or earmuffs alone. It also allows the assessment of HPDs that are worn in conjunction with helmets, eyeglasses, or other PPE, even in cases where it is not feasible to remove the PPE at the time the measurement is made.

The neck-based PAR system also incorporates a simple psychoacoustic validation procedure that can be used with this neck-based PAR measurement system (and with other free-field PAR systems that are based on behavioral threshold measures) to determine when the PAR measurement may be compromised by the ambient noise floor. Noise floor issues are a problem for REAT-based PAR systems because the noise floor can mask the test signal and artificially increase the measured threshold.

Headphone-based FAES systems can address this problem by using headphones that provide some attenuation of ambient sounds in the environment. However, FAES systems that rely on threshold measurements made in the free-field (including those made by the proposed neck-based FAES system) are unable to attenuate ambient sounds, and thus may have a difficult time measuring PAR values when they are too low to attenuate the background noise level in the room below the listener's hearing threshold.

The method proposed here requires the test administrator to position themselves near the test subject, listen to the free-field signals presented by the FAES system, and confirm audibility above the background noise level of the room. If the background noise level is similar for the experimenter and test subject, this method can help identify test results that might be distorted by background noise levels.

In the particular implementation of a neck-based FAES, the headphones placed around the neck were those from an EDARE WAHTS boothless audiometer (Kulinski and Brungart, 2022). The WAHTS headphones have the form factor of an oversized pair of wireless headphones that connect via Bluetooth to an Android tablet computer that is used to control the presentation of stimuli and collect user responses. When worn over the ears, the WAHTS headphones are capable of generating stimuli over a wide dynamic range (−10 to +70 dB HL). The large earcups also have substantial attenuation, which makes it possible to measure audiometric thresholds down to 500 Hz in most normal office environments.

The following sections describe the experiments that were conducted to validate the use of the WAHTS headphones as a neck-based PAR system.

### Calculating the Neck-to-Ear Transfer Function of the Neck-Based FAES

An obvious limitation of a neck-based FAES system is that the measurements made by such a system could vary substantially depending on how the system is placed on the neck and on the geometry of the user's head, neck, and ears. The additional distance between the earcups of the headphones and ears might also limit the maximum signal level the headphones are capable of presenting to the user. In order to address these concerns, a series of acoustic measurements were made to assess the neck-to-ear transfer function for the WAHTS headphones.

### Neck-to-Ear Transfer Function for Standard Fit on an Acoustic Manikin.

A first set of measurements was made to determine how much variation there is in the sound pressure level at the ears when the WAHTS headphones are placed across the neck. The testing was done with a GRAS 45BB-12 KEMAR acoustic manikin configured for 2-channel low-noise binaural measurements. For this testing, the outputs of the 43BB Low Noise Ear Simulator Systems in the KEMAR manikin were powered with 12HF 1-channel power modules which were attached to the inputs of an RME Babyface multichannel sound card. The WAHTS headphones were connected to an Android tablet using TabSINT software (Shapiro et al., 2019), and a Bluetooth streaming connection was used to play 21 repetitions of a diotic swept-sine test signal using the procedures (Romigh et al., 2015).

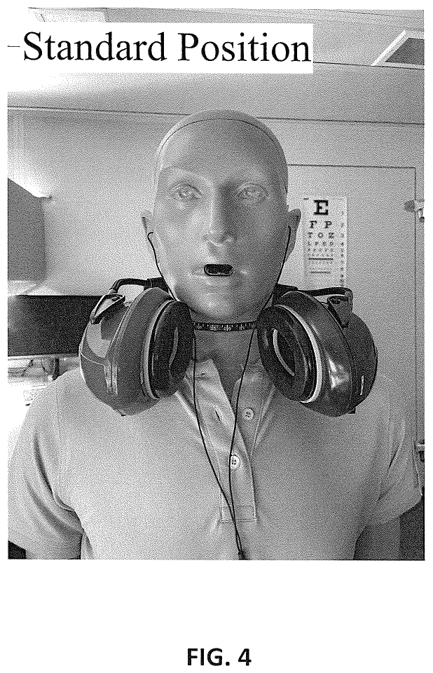

The headphones were first put on the ears of the KEMAR manikin, and a reference recording was made to determine the on-ear frequency responses of the test signal in the left and right ears of the manikin. The headphones were then removed and placed on the neck in the “standard” configuration shown in FIG. 4. Note that, in this configuration, the earcups are angled slightly away from the listener. Once the headphones were in place, the neck-to-ear transfer function was calculated by taking the difference between the frequency spectrum of the on-ear measurement and the frequency spectrum of the on-neck measurement. These headphones were then removed and replaced five more times to obtain a total of six neck-to-ear transfer functions.

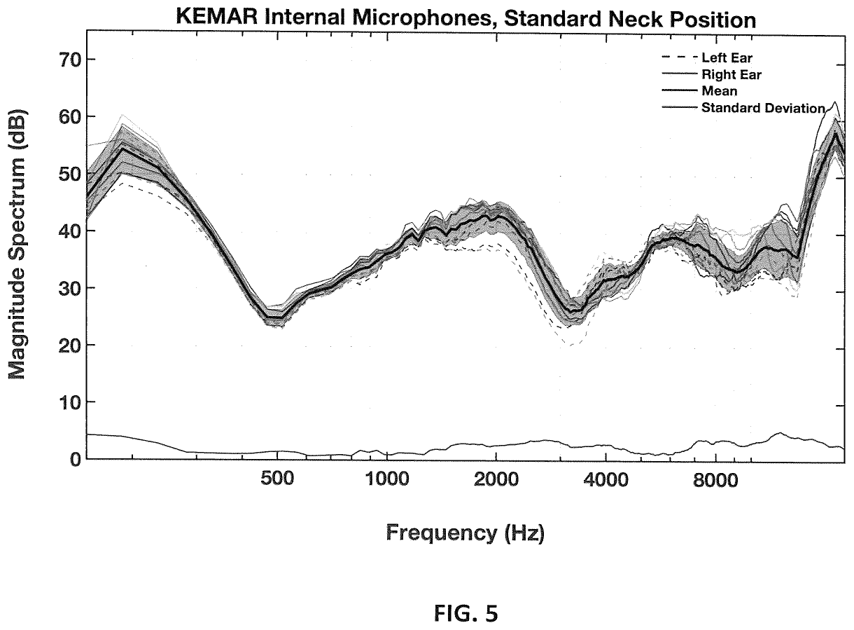

FIG. 5 shows the results of these measurements. The solid black line shows the mean value across the six placements of the headphones around the neck. As expected, the results show that the signal at the ears was always louder when the headphones were worn over the ears than when it was worn around the neck. This difference ranged from a maximum of about 55 dB at 200 Hz and 18 kHz, and a minimum of about 25 dB at 500 Hz and 3 kHz.

However, the responses were very reliable across different placements of the headphones. The gray-shaded region shows one standard deviation around the mean level at each frequency, and the red line at the bottom of the graph shows the value of the standard deviation at each frequency. The standard deviation across fittings was never more than 4 dB, and at most frequencies it was less than 3 dB. This indicates that the neck-to-ear transfer function was very reliable for repeated placements of the headphones in a standardized configuration.

### Neck-to-Ear Transfer Function for Non-Standard Fits on an Acoustic Manikin

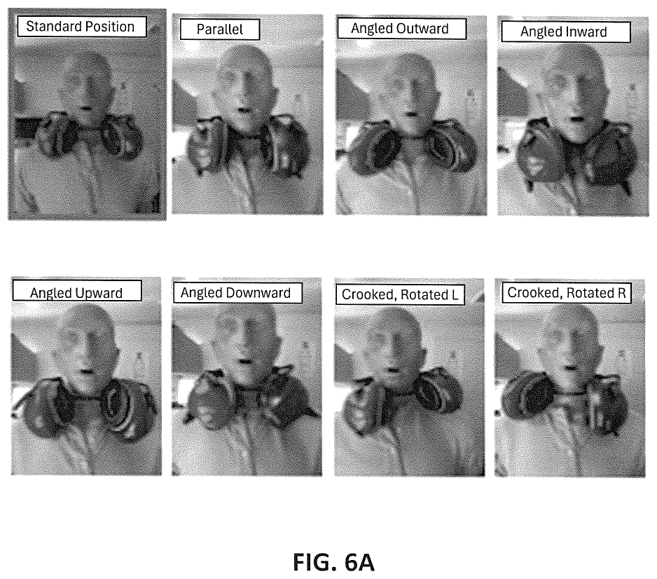

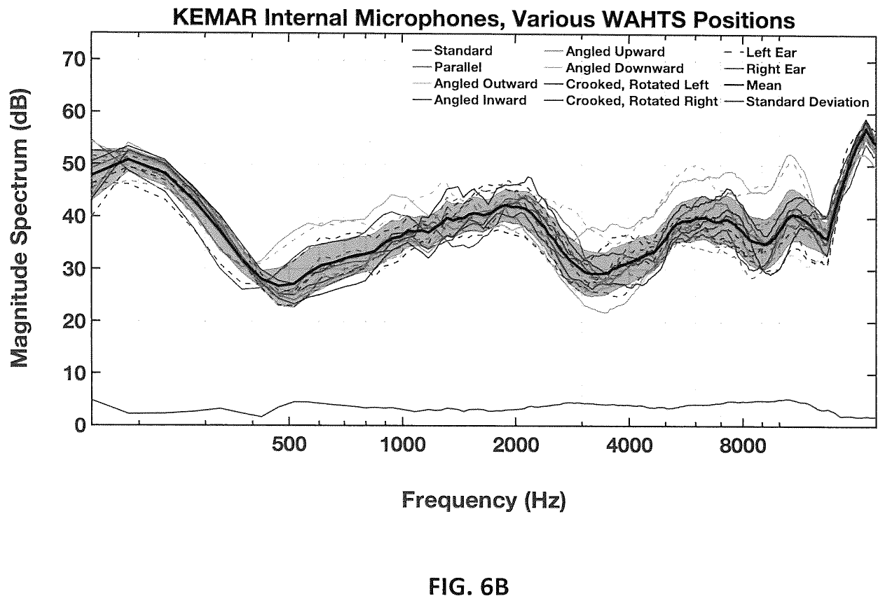

A second set of measurements was made to determine how much variability there was in the neck-to-ear transfer function when the headphones were intentionally placed in a nonstandard configuration. The measurements used the same procedure described in the prior section, but the headphones were placed on the neck in the standard configuration, with the headphones angled away from the neck, and with 7 other non-standard configurations as illustrated by the 8 images in FIG. 6A.

The results of these measurements are shown in FIG. 6B. The results show that the largest deviations from the standard fitting occurred when the headphones were angled up (resulting in a larger than standard signal at the ears) and when the earcups were angled down (resulting in a smaller than standard signal in the ears). Even with these outliers, the neck-to-ear transfer function was relatively stable, with a standard deviation less than 5 dB across all the frequencies tested. These results indicate that the neck-to-ear transfer function should be relatively stable if reasonable care is taken to ensure the headphones are placed on the neck in the standard configuration.

### Neck-to-Ear Transfer Functions Variability across Individual Human Listeners.

The third set of measurements was made to determine how much variability there is in the neck-to-ear transfer function on human subjects. These measurements were made using the same procedures used with the KEMAR, except that the in-ear microphones were replaced with SP-TFB-2 low noise in-ear Binaural microphones (Sound Professionals, Hainesport, NJ) that were held in the conchae of the subjects with a plastic hook. Three measurements were made for each of 7 subjects (3 M, 4 F) ranging in height from 145 cm to 183 cm (σ=12.2 cm).

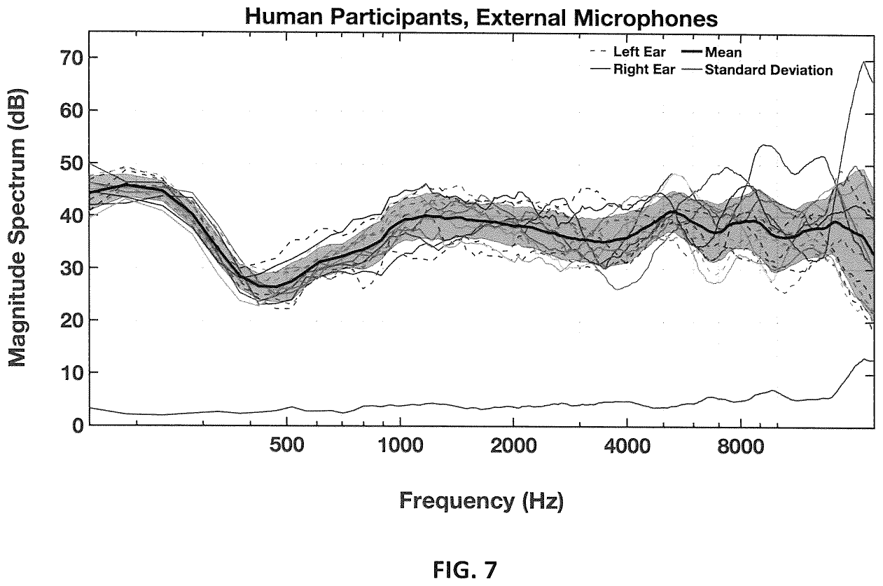

The three neck-to-ear transfer functions for each subject were averaged together to obtain the individual left-ear and right-ear responses in FIG. 7. The results show a neck-to-ear pattern that is very similar to that obtained with the KEMAR manikin, with a minimum value of approximately 30 dB at 500 Hz and a stable value of approximately 35-40 dB from 1 kHz to 3 kHz. Again, the standard deviation across measurements was less than 5 dB across all frequencies less than 3 kHz.

### Experimental Validation of Neck-Based Fit Test

The results of the acoustic measurements in the prior section suggest that the neck-to-ear transfer function is reasonably stable across placement on different listeners and across repeated placements on the same listener. This suggests that it may be possible to use neck-mounted headphones to get a reliable measure of PAR in situations where it is not feasible to place the headphones over the HPDs. In order to further test this possibility, an experiment was conducted to compare the results using the neck-mounted PAR method to a current commercially available MIRE-based field attenuation evaluation system.

### Methods

Participants. A total of 32 subjects volunteered to participate in the study after completing their annual hearing testing in the Walter Reed hearing conservation clinic. Audiometric pure tone threshold information from these hearing tests were available for 26 of the 32 participants. This information was collected using an automated audiometer (CCA-200, Benson Medical Instruments, Minneapolis, MN). Age and sex information was also available for these 26 participants. Seventeen of these 26 participants were male, and their average age was 33.8 years (std. dev. 9 years).

Neck-Par System. The system used to conduct the Neck-Par procedure consisted of a WAHTS boothless audiometer (Edare, Lebanon, NH and Kulinski and Brungart, 2022) that was connected to a tablet PC running TABSint test software (Shapiro et al., 2019).

The WAHTS headphones were configured to conduct 3-frequency hearing threshold tests using a modified Bekesy tracking technique. The stimuli used for the modified Bekesy tracking consisted of ⅓rd octave bands of noise centered at 500 Hz, 1 kHz, and 2 kHz, in accordance with the bandwidth specifications in ANSI (2018).

The narrowband noises were presented simultaneously from both ears, but the noises presented from the two ears were statistically independent to approximate a diffuse field at the location of the listener's ears. At the start of each threshold measurement, a pulsed narrowband noise signal was presented above threshold and the listener was told to hold down a button on the tablet screen whenever the sound was loud enough to be heard and to release the button whenever the sound became too quiet to be heard.

Then, upon the first button press, the level of the pulsed sound started to decrease 2 dB with each 700 ms pulse of noise until the listener released the button to indicate the sound was no longer audible. Then the sound began to increase 2 dB with each 700 ms pulse of noise until the listener pressed the button to indicate it was audible again. This process repeated until the listener completed a total of 6 reversals. The threshold was then estimated from the average of the last reversal points.

Procedure. The first step of the procedure was to use the WAHTS audiometer to collect an on-ear unoccluded threshold. The WAHTS headphones were placed on the ears normally, and the modified Bekesy procedure was used to obtain on-ear threshold measurements at 500 Hz, 1 kHz, and 2 kHz.

The second step was to obtain an on-neck unoccluded threshold. The WAHTS headphones were placed around the neck in the standard configuration shown in FIG. 4 and threshold were obtained at 500 Hz, 1 kHz, and 2 kHz.

The third step was to obtain on-neck threshold values in a “standard” earplug condition. In this condition, the experimenter first used an Eargauge earplug sizing tool (3M, St. Paul, MN) to select the correct size (small, medium, or large) for a triple-flanged plug. Listeners then inserted triple-flange probed earplugs of the appropriate size into the ears. These triple-flanged plugs were specially modified surrogate earplugs that were penetrated by a probe tube for use with a commercially-available microphone-in-real-ear FAES System (EarFit, 3M, St. Paul, MN).

The EarFit was then used to calculate a binaural PAR-84 value for the surrogate earplugs, which it does by playing a broadband sound through a loudspeaker and comparing the signal level inside and outside of the earplug in each ear. The experimenter manually recorded this PAR value. The plugs were then left in place, and the WAHTS headphones were used to obtain on-neck occluded threshold values for the triple-flange plugs at 500 Hz, 1 kHz, and 2 kHz.

The fourth step was to obtain on on-neck threshold values in a “double” protection condition. In this condition, an earmuff hearing protector (ComTac III, 3M, St. Paul, Mn.) was placed on the head over the standard triple-flanged earplug, and the WAHTS headphones were used to collect on-neck occluded thresholds at 500 Hz, 1 kHz, and 2 kHz.

The fifth step was to obtain on-neck threshold values in a “compromised” hearing protection condition. In this condition, scissors were used to cut off the first (smallest) flange of the triple-flanged earplugs used in the third step of the procedure. These compromised earplugs were inserted into the ears, and a PAR value was measured using the EarFIT system. Then the WAHTS headphones were used to obtain on-neck occluded threshold values for the compromised triple-flanged plugs at 500 Hz, 1 kHz, and 2 kHz.

### Results

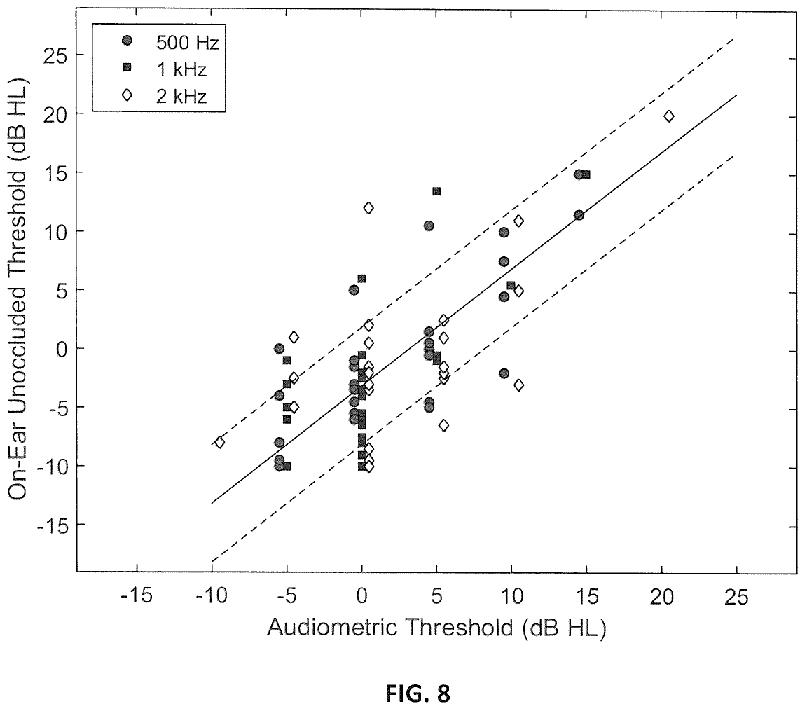

Comparison of On-Ear WAHTS Thresholds. FIG. 8 shows a comparison between the on-ear unoccluded thresholds measured using the modified Bekesy method for ⅓rd octave bands a 500 Hz, 1 kHz, and 2 kHz and the corresponding pure-tone thresholds for the 26 subjects who also had audiometric data available from their annual hearing tests.

The mean threshold value for the narrow-band noise thresholds was 3.15 dB higher than the mean threshold for the pure-tone thresholds. The standard deviation of the difference between the two thresholds was 4.8 dB. Although there were a few outliers, these results are comparable to those obtained in a recent meta-analysis that compared manual and automated audiometric thresholds measurements for 766 subjects across 10 studies (Mahomed et al., 2022). That study found a standard deviation of 5.3 to 5.5 dB between the manual and automated threshold measurements at 500 Hz, 1 kHz, and 2 kHz.

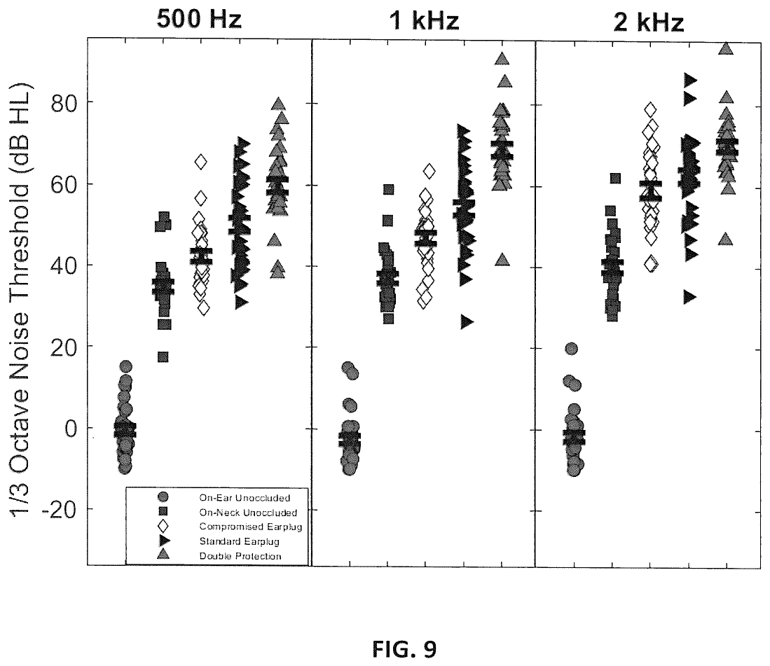

Comparison of Thresholds across Test Conditions. FIG. 9 shows the absolute thresholds measured for the ⅓ octave noises at each frequency for each of the five conditions of the experiment. The error bars in each column represent the 95% confidence intervals around each data point. These confidence intervals indicate that the mean thresholds in each condition were significantly different from one another at the p<0.05 level. Thus, it appears that, even without correcting for individual hearing thresholds, the on neck method was sensitive to differences in the attenuation values provided by different levels of hearing protection.

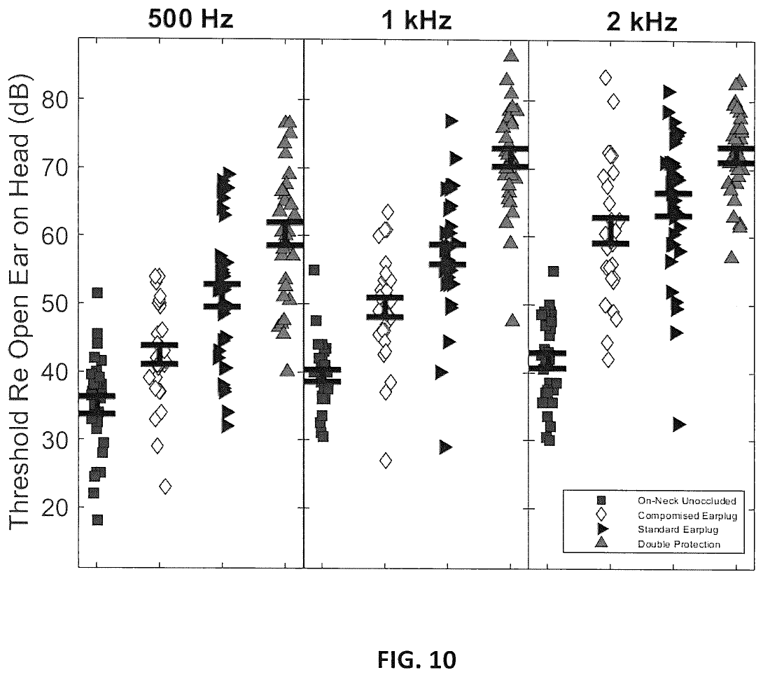

Comparison of Uncorrected Estimated Attenuation Values across Test Conditions. FIG. 10 shows the uncorrected estimates of hearing protection attenuation obtained for each hearing protection device at each frequency. Each dot represents the difference between the on-neck threshold for a given hearing protection device at a given frequency and the on-ear threshold obtained for that same listener at that same frequency.

The error bars in each column represent the 95% confidence intervals around each data point. These confidence intervals indicate that the mean estimated attenuations in each condition were significantly different from one another at the p<0.05 level. However, these attenuation values are not meaningful without making a correction for the difference between the signal that reaches the ears in the on-ear condition and the signal that reaches the ears in the on-neck condition at each frequency. This correction factor is crudely approximated by the mean attenuation value obtained for the on-neck unoccluded condition (blue diamonds).

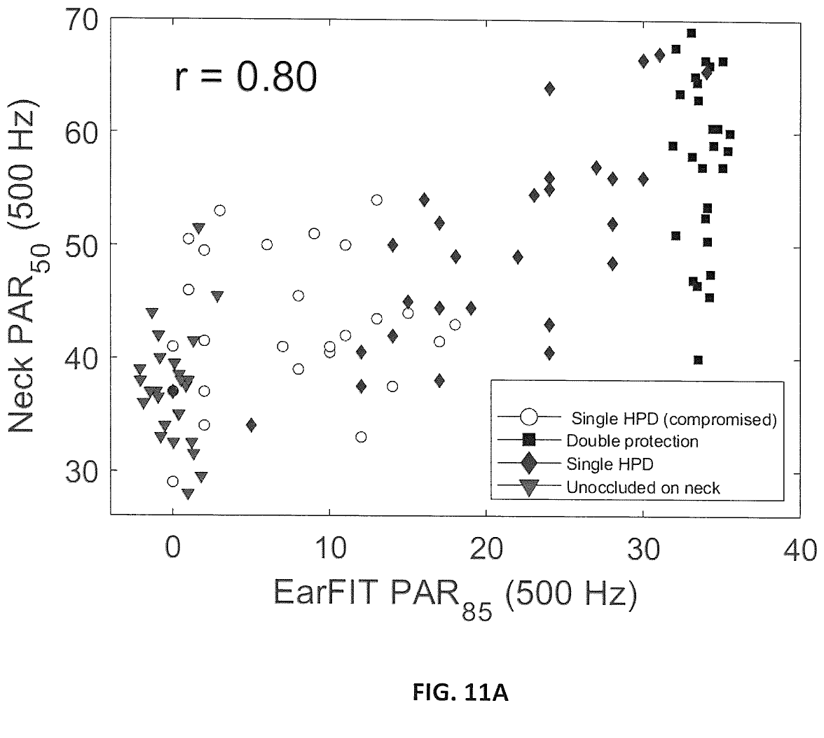

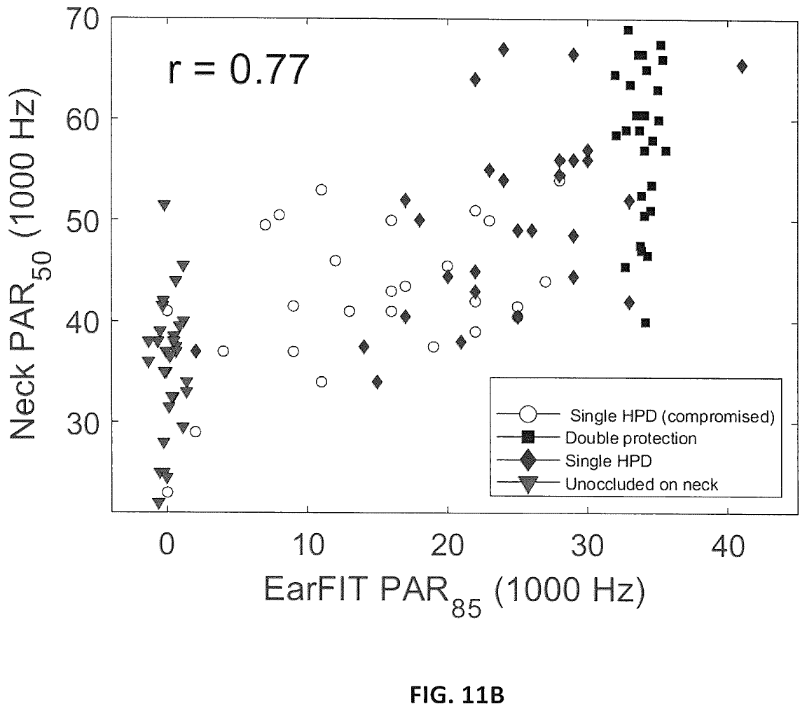

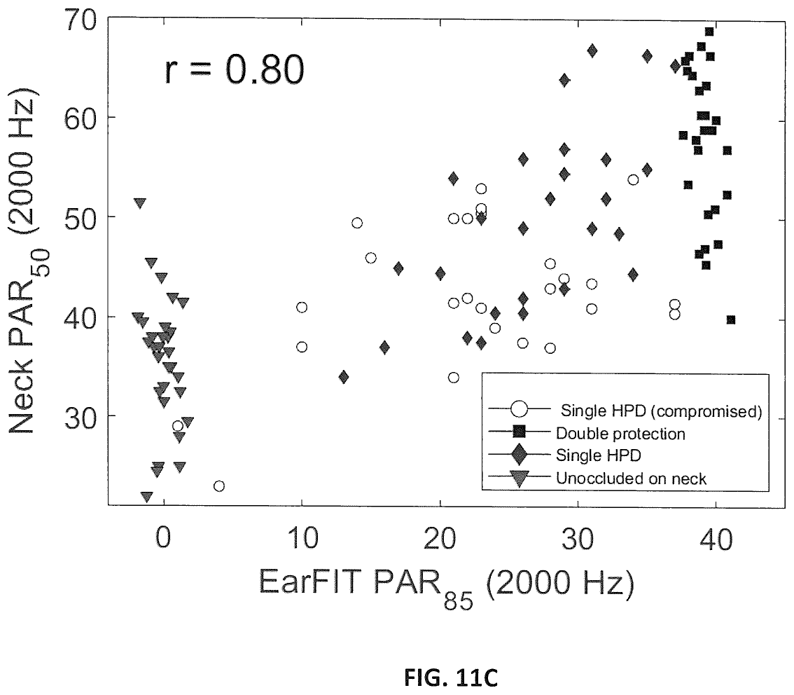

Comparison of Frequency-dependent Attenuation between the Neck-based System and the EarFIT. The ultimate goal of the neck-based system is to obtain accurate estimates of the attenuation that a hearing protector provides at each frequency. In order to evaluate its effectiveness for making these measurements, the attenuations estimated by the neck-based PAR system at each frequency were compared to PAR estimates provided by the 3M EarFIT system for the same earplug FIT. FIGS. 11A-11C are plots of the unadjusted on-neck attenuation estimations, obtained by subtracting on-ear thresholds from the on-neck thresholds, as a function of the individual frequency PAR84 values measured by the EARFit system.

The open-ear conditions were assigned a nominal PAR value of 0 dB at all frequencies, and a Gaussian jitter with a 1 dB standard deviation was added to make the individual data points easier to see on the plot. No EarFIT PAR values were available for the double-protection conditions, so they were assigned nominal PAR84 values of 34 dB at 500 Hz, 39 dB at 1 kHz, and 39 dB at 2 kHz, also with a Gaussian jitter with a 1 dB standard deviation.

These values were estimated by adding 5 dB to the mean attenuation with a 3M X5 earmuff (Luciana Macedo, 2016), and subtracting 6 dB to account for the conversion from PAR50 to PAR84. Note that the addition of 5 dB to the attenuation value of an earmuff to account for the additional attenuation provided by double hearing protection is a commonly used rule of thumb in audiology that is included in the OSHA technical manual (Safety et al., 1995).

Although there is some spread in the data, the results shown in FIGS. 8A-8C do clearly show a systematic relationship between the unadjusted attenuations estimated from the on-neck system and the actual PAR84 values measured by the EarFIT system. The correlation coefficients ranged from 0.77 to 0.8 across the three frequencies.

Selection of optimal frequency-dependent correction factors. The underlying assumption of the neck-based PAR system is that the level of the signal that reaches the ears from a headset worn around the neck differs from the level of the signal that reaches the ears when the headset is worn over the head by a “correction factor” that differs across frequencies but is relatively stable across different fittings of the headset on different listeners.

To identify the optimal correction factors for each frequency, MATLAB was used to perform an exhaustive search to identify the offset values at 500 Hz, 1000 Hz, and 2000 Hz that resulted in the best FIT between the PAR values obtained with neck-based system and the PAR values that were measured with the EarFIT. The full-factorial search used a 1 dB step size and included all correction factors between 25 and 40 dB at 500 Hz, between 30 and 50 dB at 1 KHz, and between 30 and 50 dB at 2 kHz.

The analysis excluded the double hearing protection condition, which did not have any measured PAR values on the EarFIT, and any conditions where the experimenter indicated they were unable to hear the test signal during the on-neck PAR measurement. Open-ear measurements were included, with the EarFIT PAR value set to 0 dB. This resulted in a total of 108 data points that were included in the search procedure.

The optimization was based on two factors. The first was the percentage of trials where the neck-based PAR estimate was within 5 dB of the measured PAR84 value, and the percentage of trials where the PAR estimate was within 10 dB of the measured PAR84 value.

Of all the combinations tested, the one with the best overall fit has a correction value of 35 dB at 500 Hz, 42 dB at 1 kHz, and 41 dB at 2 kHz. These values produced an estimated PAR value that was within 5 dB of the measured EarFIT value for 76% of the measurements, and within 10 db of the measured EarFIT value for 97% of the measurements.

It is also notable that these values are similar to the average differences between the On-Neck Unoccluded and On-Ear Unoccluded values at each frequency shown in FIG. 11. The correction values are also similar to, but slightly larger than, the 28 dB, 38 dB, and 38 dB values at 500 Hz, 1 kHz, and 2 kHz that were obtained from the on-neck to on-ear transfer functions that were measured on human subjects (FIG. 7). The 3-7 dB difference between these measured values and the best-fitting values may be related to the roughly 6 dB difference between the PAR84 value on the EarFIT and the mean value of the PAR (PAR50).

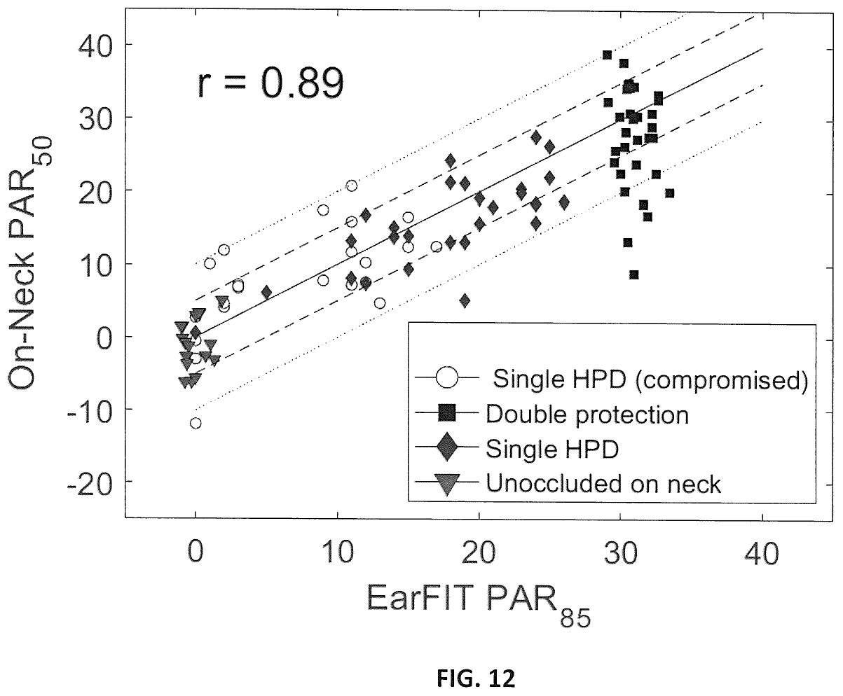

Estimation of PAR value from Neck-Based System. FIG. 12 shows the estimated on-neck PAR84 values for each data point in the validation experiment as a function of the measured binaural PAR84 obtained from the EarFIT system. These data show that the neck-based PAR system was able to obtain a reasonably accurate PAR value in the vast majority of the tests.

Note that the double protection conditions (black squares) were not measured with the EarFIT, so incorrect estimates for those data points do not reflect errors neck-based PAR system. Excluding those data points, only 4 out of 126 PAR estimates were more than 10 dB from the measured value, and none were more than 15 dB from the measured value. The correlation between measured and estimated PAR values was 0.89.

Cross-Validation of Neck-Based PAR. The values shown in FIG. 11 suggest a strong correlation between the estimated and measured PAR values for the neck-based PAR system. However, this data reflects offset parameters that fit to the same data set used to validate the system, which might result in an overestimate of the system's measurement accuracy.

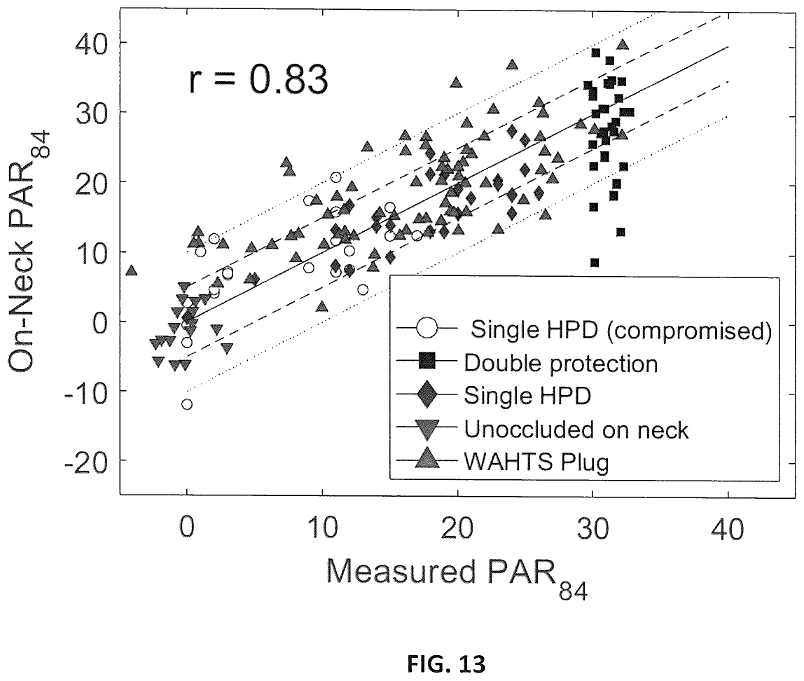

FIG. 13 shows the same figure as FIG. 12 but with an additional 78 data points added from a study that compared the neck-based PAR values obtained with foam earplugs with PAR values that were measured from the difference between the occluded and unoccluded thresholds measured with the WAHTS headphones (using the procedures in Kulinski and Brungart, 2022). The WAHTS headphones thresholds were converted from PAR50 estimates to PAR84 estimates by subtracting 6 dB.

However, the same 35 dB, 42 dB, and 41 dB correction values at 50 Hz, 1 kHz, and 2 kHz were used for both the new and old data sets. Although the measurements obtained with the additional data were not quite as accurate as those obtained with the original data set, the overall fit was still remarkably good, with 66% of the estimates within 5 dB and more than 90% within 10 dB. If these metrics are applied only to the data from the new study, they show that 59% of the estimates were within 5 dB and 85% were within 10 dB, with a correlation coefficient r of 0.71.

### Psychoacoustic Validation of Test Environment

One downside of all FAES that use the REAT method to measure the PAR in the free-field is that they are very sensitive to ambient noise levels, particularly when they are being used to test HPDs that only provide a small amount of attenuation. When an HPD is producing 0 dB or near 0 dB of attenuation, the occluded threshold will be the same as the open ear threshold, and the ambient noise level at that frequency has to be quiet enough to make an absolute threshold measurement in order to obtain an accurate estimate of attenuation.

Otherwise, the ambient noise will mask the target signal, which will result in an elevated occluded threshold measurement and an overestimate of the PAR. The Occupational Safety and Health Administration recommends a Maximum Permissible Ambient Noise Level (MPANL) of 40 dB SPL for making pure tone threshold measurements at 500 Hz, which is much less stringent then the MPANLs recommended by the American National Standards Institute or the National Hearing Conservation Association (Lankford et al., 1999) but still somewhat difficult to achieve outside of a sound-treated booth.

In this study, the test administrators were instructed to stand near the subject being tested and carefully listen to the one-third octave Bekesy tracks that were used to estimate the threshold at 500 Hz, 1 kHz, and 2 kHz. Because Bekesy tracks play noise continuously, there is a very easy way for the test-administrator to determine if the threshold measured by the Bekesy track was above the ambient noise level.

If the signal the test administrator hears when the threshold is administered is continuous then it is very likely that the threshold was not affected by the ambient noise level and that the PAR measurement will be reliable. This is true because it means that the level of the signal at its quietest point (the point where the subject stopped hearing the signal and stopped pressing the button) was loud enough that it still could be heard above the noise floor at the location of the test administrator (whose ears are likely to be at least as far from the neck-based headset as the subject's ears).

In contrast, if the test administrator hears the Bekesy noise track disappear below the noise floor in the room (i.e., they hear the signal get quiet, then a gap in time before they start hearing it again on the rising portion of the Bekesy track), then it means that the ambient noise level was loud enough that it may have masked the Bekesy track at threshold. This does not necessarily mean that the PAR measurement was invalid, as it is possible because of the closer proximity to the headphones for the signal to be audible to the subject but not to the test administrator. In such a case, the PAR value measured can be viewed as an upper limit, with a reasonable assumption that the PAR is less than or equal to that value.

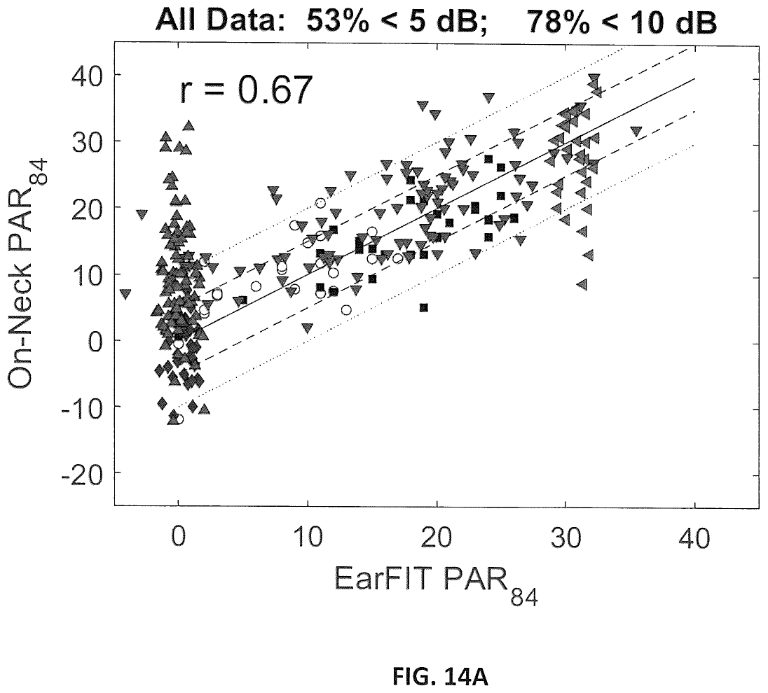

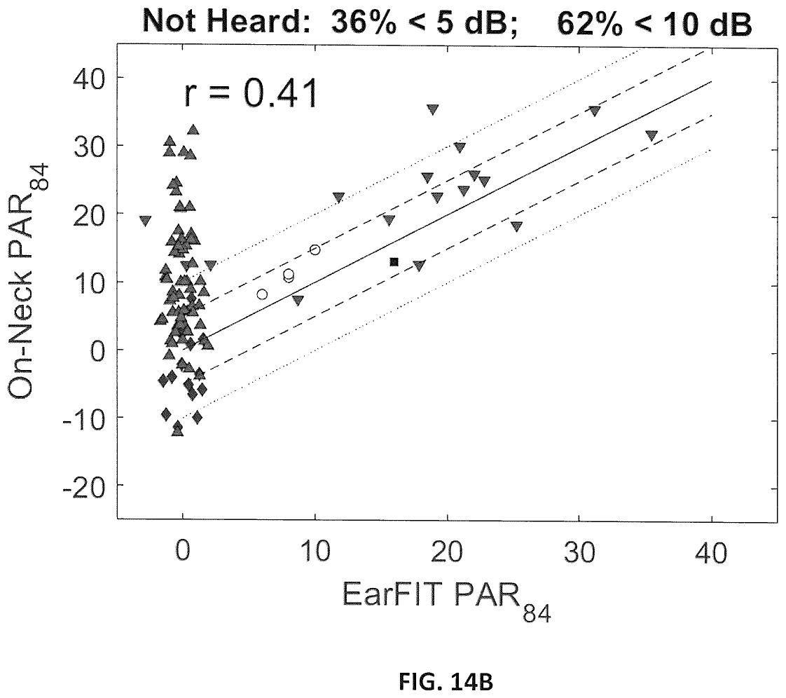

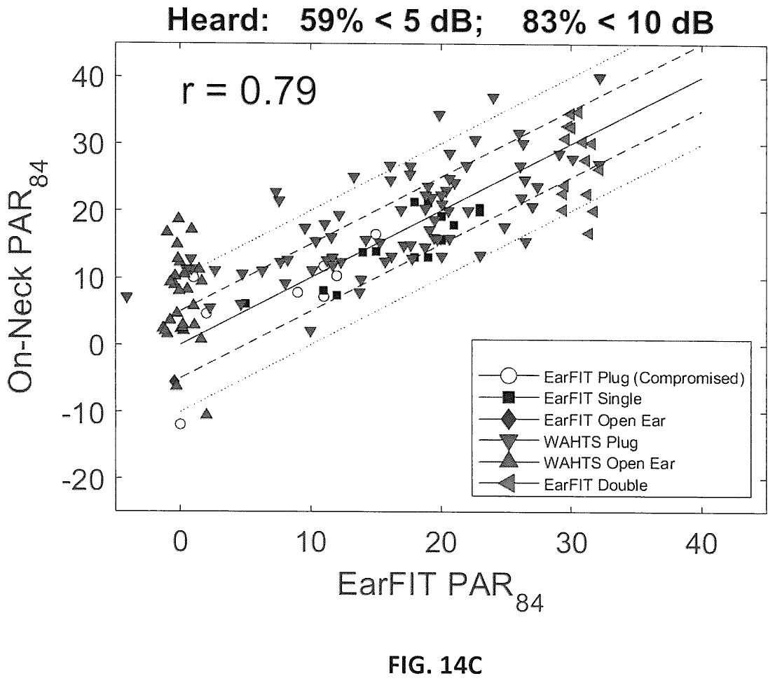

FIGS. 14A-14C show the effect these validation questions had on the results of the on-neck testing. FIG. 14A shows the results from all 335 trials tested, regardless of whether the test administrator heard the test stimuli. FIG. 14B shows the results for the 103 trials where the administrator indicated they did not hear all three Bekesy tracks. FIG. 14C shows the results for the 145 trials where the test administrator said they did hear all three trials (there were 87 trials at the start of the study where the administrator was not asked about hearing the levels).

Comparing the right two panels, it is clear that the PAR estimates where substantially more accurate in trials where the administrator reported hearing the trials. In particular, there were a large number of trials in the “Open Ear” condition where the stimuli were not heard where the PAR was overestimated by a wide margin, in some cases as much as 35 dB. The psychophysical validation performed by the experimenter was able to eliminate these trials, and increase the overall percentage of estimates within 5 dB or 10 dB by 5 percent.

While not perfect, this method can clearly increase the reliability of the On-Neck PAR estimates. As is suggested by the data, this psychophysical validation is most important in cases where the hearing protector is providing little or no attenuation. As the attenuation of the protector increases, the probability of interference from the ambient noise level decreases. In our data set, there were no cases where the experimenter reported not hearing the sound in the double protection condition.

### Advantages and New Features

Hearing protector fit testing is considered a best practice for hearing conservation and is now required by DoD Instruction 6055.12 for all US Service Members who are exposed to 8-hour average noise levels greater than 95 dBA SPL. However, most commercially-available FAES systems are only capable of measuring PAR with Earplug-based HPDs. Very few are capable of measuring PAR with Earmuffs, and none are capable of measuring PAR with double hearing protection or with an individual's own Earmuff devices.

Here we have described a new technique for measuring PAR that uses a standard calibrated headphone placed around the neck to measure PAR values for HPDs placed on the individual's ears. This technique can be used for any HPD device, including earplugs. However, when the goal is to estimate the PAR for earplugs it would generally make more sense to make the occluded threshold measurements with the headphones placed over the ears, rather than on the neck. Placing the headphones over the earplugs provides greater attenuation of ambient sounds that could interfere with the threshold measurement and requires less acoustic output to measure PAR for devices with a great deal or attenuation or for individuals who have significant hearing losses.

However, when the goal is to measure PAR value for Earmuffs or Double Hearing Protection, there are many situations where the neck-based PAR system may be the only viable option. The current gold standard for making measuring PAR with these devices would require a sound-treated booth with a diffuse noise field, something which will almost never be available in locations where PAR measurements need to be made on noise-exposed workers. The neck-based PAR system makes it possible to estimate PAR for Earmuffs and Double Hearing Protection in almost any environment.

The psychoacoustic validation method also provides a simple but effective way to improve the reliability of the system by identifying threshold measurements that are unlikely to have been compromised due to interference from the ambient noise level in the room. Both the test administrator and the subject are located in the same ambient noise field, and if the administrator is able to hear the entire track of the Bekesy threshold measurement from a location next to the subject, it is unlikely that ambient noise had an influence on the threshold measured for the subject at that frequency.

If the administrator does not hear the ambient noise track, then the earplug fit can be adjusted and the measurement can be repeated. The probability that the experimenter will be able to hear the Bekesy threshold track increases with the attenuation of the tested HPD, so if the goal of the fit-check procedure is to achieve a reasonable minimum PAR value for all subjects, it is likely that the administrator will be able to use psychoacoustic validation to verify the Bekesy threshold tracks for all measurements where the subject is achieving an acceptable PAR.

In summary, the main advantages of the neck-based PAR system are as follows.

The neck-based PAR system provides the only viable method for measuring PAR values in field conditions when listeners are wearing double hearing protection.

The neck-based PAR system provides the only viable method for measuring PAR values in field conditions when listeners are wearing their own earmuffs (thus making use of an instrumented ear cushion impractical), or when they are wearing personal protective equipment that might impact the PAR value but cannot be worn underneath headphones (thus making FAES systems based on earphones worn over the HPD impractical).

The neck-based PAR system provides compatibility with PPE also makes it possible to use the neck-based PAR to get an estimate of the PAR for individuals under field conditions without removing any PPE. This can be done by making a neck-based threshold measurement, applying the frequency-dependent correction factors, and comparing it to the on-ear measurement made at an earlier or later time. The ability to make PAR measurements under these conditions make the neck-based PAR system very useful for conducting range inspections or other inspections of how hearing protection is being used under real world conditions.

The neck-based PAR system can be implemented by modifying existing headphone-based PAR systems, and provides the flexibility of switching between neck-based and ear-based PAR measurements with minimal changes in software and equipment. This also makes it very portable and suitable for field use.

The use of Bekesy tracking to measures thresholds increases the accuracy of the threshold measurements and makes it possible for the test administrator to monitor the test stimuli and ensure they are audible over the ambient noise floor. Other types of threshold tracking (Hughson-Westlake, for example) would not present stimuli at predictable levels and times and that would not allow this psychoacoustic validation to take place.

Alternatives The neck-based PAR system also makes it possible to make these measurements with the same equipment that is currently used in FAES systems to make over-the-ear PAR measurements on Earplugs. The validation study presented here used the WAHTS portable audiometer, but there is no reason to believe that similar results could not be achieved with other headphone systems either without modification or with slight modifications to increase the maximum acoustic output and make it easier to place the headphones around the neck. In the context of this disclosure, an audiometer can be assumed to be any system capable of creating a calibrating audio signal over headphones, adjusting that level of that output in a systematic fashion, and using behavioral responses or biomedical sensors to estimate the lowest level at which the audio signal is detectable by the listener.

It would only be necessary to make new measurements to determine the neck-to-ear transfer correction values for these other headphone devices. It would also be possible to implement the system using the high-frequency headphones that are distributed with most clinical audiometers. Wearing the audiometer headphones around the neck would make it possible to make calibrated measurements of the occluded thresholds using standard audiometric measurement techniques.

Note that, in this application, the subject is likely to be in a sound-treated booth. This would reduce the ambient noise level and might make it possible to use the open-ear on-neck threshold as a comparison point rather than the on-ear threshold, which would reduce any variability that might occur across different placements of the headphones around the neck on different listeners.

Note that an examination of the prior art has not revealed any prior discussion of the possibility of using headphones worn around the neck as a way to get a reliable threshold at ear level. An electronic search on this topic reveals several discussions indicating that this technique is not recommended as a way to listen to music (due to decreased sound quality) but no discussion of the possible advantages of this technique. We believe that the high level of reliability we were able to achieve for the headphone-to-ear transfer function is a new discovery and that it will surprise many experienced practitioners in the field. Recent patent applications in this area have focused either on using neural networks to estimate PAR values from pictures of earplug fittings (Ser. No. 16/797,554) or using transducers in the ear or in the earcup to make the measurements (Ser. No. 18/096,549).

Many modifications and other embodiments will come to the mind of one skilled in the art having the benefit of the teachings presented in the foregoing descriptions and the associated drawings. Therefore, it is understood that the foregoing is not to be limited to the example embodiments, and that modifications and other embodiments are intended to be included within the scope of the appended claims.

### REFERENCES

Agency, E. P. (1979). Code of federal regulations 40 cfr 211 subpart b section 221.207. https://www.ecfr.gov/current/title-40/chapter-I/subchapter-G/part-211/subpart-B/section-211.207.
ANSI, A. (2018). S12.71-2018, performance criteria of systems that estimate the attenuation of passive hearing protectors for individual users. New York: American National Standards Institute.
ANSI, A. (2020). S12.6-2016 (r2020), methods for measuring the real-ear attenuation of hearing protectors. New York: American National Standards Institute.
Berger, E., Kieper, W., and Gauger, D. (2003). Hearing protection: Surpassing the limits to attenuation imposed by the bone-conduction pathways. Journal of the Acoustical Society of America, 114:1955-1967.
Federman, J., Karch, S. J., and Duhon, C. (2021). How hearing conservation training format impacts personal attenuation ratings in us marine corps training recruits. International Journal of Audiology, 60(2):151-159.
Kulinski, D. and Brungart, D. S. (2022). Using a boothless audiometer to estimate personal attenuation rating in a military hearing conservation clinic. JASA Express Letters, 2(4).
Lankford, J. E., Perrone, D. C., and Thunder, T. D. (1999). Ambient noise levels in mobile audiometric testing facilities: compliance with industry standards. AAOHN Journal, 47(4):163-167.
Lempert, B. L. and Edwards, R. G. (1983). Field investigations of noise reduction afforded by insert-type hearing protectors. American Industrial Hygiene Association Journal, 44(12):894-902.
Luciana Macedo, Terry Gorman, E. H. B. (2016). Assessment of the effects of various personal protective equipment (ppe) and apparel in the performance of earmuffs. In AUSTRALIAN INSTITUTE OF OCCUPATIONAL HYGIENISTS, 34th ANNUAL CONFERENCE EXHIBITION RACV ROYAL PINES RESORT|GOLD COAST, QUEENSLAND 3-7 Dec. 2016, pages 151-159.
Mahomed, F., Swanepoel, D. W., Eikelboom, R. H., and Soer, M. (2022). Validity of automated threshold audiometry: a systematic review and meta-analysis. Ear and hearing.
Murphy, W. J., Gong, W., Karch, S. J., Federman, J., and Schulz, T. Y. (2022). Personal attenuation ratings versus derated noise reduction ratings for hearing protection devices. The Journal of the Acoustical Society of America, 152(2):1074-1089.
of Health, U. D., Services, H., et al. (1998). Criteria for a recommended standard: Occupational noise exposure: Revised criteria (dhhs [niosh] publication no. 98-126). Cincinnati,
OH: Centers for disease control and prevention. National Institute for Occupational Safety and Health.
OSHA, U. (2013). Osha technical manual (otm) section iii: Chapter 5—noise. Department of Labor.
Romigh, G. D., Brungart, D. S., and Simpson, B. D. (2015). Free-field localization performance with a head-tracked virtual auditory display. IEEE Journal of Selected Topics in Signal Processing, 9(5) 943-954.
Safety, U.S.O., of Science, H.A.O., and Assessment, T. (1995). OSHA Technical Manual, volume 1. US Department of Labor, Occupational Safety and Health Administration . . . .
Shapiro, M. L., Norris, J. A., Wilbur, J. C., Brungart, D. S., and Clavier, O. H. (2019).
Tabsint: open-source mobile software for distributed studies of hearing. International Journal of Audiology, pages 1-8.

1. A method for determining a personal attenuation rating of a hearing protection device on a listener using a neck-based PAR system, the method comprising:
placing headphones with an audiometer around ears of the listener;
operating the audiometer to measure an on-ear hearing threshold of the listener without the hearing protection device;
placing the headphones with the audiometer around a neck of the listener;
operating the audiometer to measure an on-neck hearing threshold of the listener with the hearing protection device;
applying a neck-to-ear transfer function to a difference in the measured on-ear and on-neck hearing thresholds to determine a measured personal attenuation rating of the hearing protection device;
determining if an ambient noise level in an area surrounding the listener compromised the measured personal attenuation rating;
if the ambient noise level in the area did not compromise the measured personal attenuation rating, then the measured personal attenuation rating is reliable; and
if the ambient noise level in the area did compromise the measured personal attenuation rating, then the measured personal attenuation rating is not reliable.

2. The method according to claim 1 wherein a test administrator in the area verifies that the ambient noise level did not compromise the determined personal attenuation rating in response to hearing an audible test signal from the audiometer at a lowest level tested when measuring the on-neck hearing threshold.

3. The method according to claim 1 wherein the hearing protection device worn by the listener comprises ear muffs, and wherein a size of the headphones with the audiometer will not fit over the earmuffs.

4. The method according to claim 1 wherein the hearing protection device worn by the listener comprises both ear plugs and ear muffs, and a size of the headphones with the audiometer will not fit over both the ear plugs and the earmuffs.

5. The method according to claim 1 further comprising determining the neck-to ear transfer function based on the following steps:
placing headphones with an audiometer around the ears of an acoustic manikin;
operating the audiometer to generate a test signal, with the test signal being used to determine on-ear frequency responses in the ears of the acoustic manikin;
placing the headphones with the audiometer around the neck of the acoustic manikin;
operating the audiometer to generate the test signal, with the test signal being used to determine on-neck frequency responses in the ears of the acoustic manikin; and
determining a difference between the frequency spectrum of the on-ear measurements and the frequency spectrum of the on-neck measurements to determine the neck-to-ear transfer function.

6. The method according to claim 1 wherein the listener operates the audiometer.

7. The method according to claim 1 wherein the audiometer is configured to generate tones and is coupled to a tablet computing device that includes a touch screen, and wherein the listener operates the audiometer via the touch screen.

8. The method according to claim 7 wherein the audiometer and the tablet computing device are wirelessly coupled together.

9. The method according to claim 7 wherein the touch screen is configured to provide a response button, and wherein the listener operates the audiometer holding down the response button when the tone is heard, and releasing the response button when the tone is no longer heard.

10. The method according to claim 1 wherein the headphones comprise spaced apart earcups, with the earcups being angled away from the listener when the headphones are placed around the neck of the listener.

11. A neck-based personal attenuation rating (PAR) system comprising:
headphones with an audiometer;
a computing device comprising:
a touchscreen with a response button to be operated by the listener to determine an on ear threshold while the headphones with the audiometer are placed
over ears of a listener not wearing a hearing protection device, and an on neck threshold while the headphones with the audiometer are placed around a neck of the listener while wearing the hearing protection device;
a memory configured to store the on ear threshold and the on neck threshold, and
a processor coupled to the touchscreen and to the memory and configured to apply a transfer function to a difference between the on ear threshold and the on neck threshold to determine a personal attenuation rating of the hearing protection device; and
an ambient noise level detector configured to determine if an ambient noise level in an area surrounding the listener compromised the determined personal attenuation rating.

12. The neck-based personal attenuation rating (PAR) system according to claim 11 wherein the ambient noise level detector is a test administrator in the area to verify that the ambient noise level did not compromise the determined personal attenuation rating in response to hearing an audible test signal from the audiometer at a lowest level tested when measuring the on-neck hearing threshold.

13. The neck-based personal attenuation rating (PAR) system according to claim 11 wherein the hearing protection device worn by the listener comprises ear muffs, and wherein a size of the headphones with the audiometer will not fit over the earmuffs.

14. The neck-based personal attenuation rating (PAR) system according to claim 11 wherein the hearing protection device worn by the listener comprises both ear plugs and ear muffs, and a size of the headphones with the audiometer will not fit over both the ear plugs and the earmuffs.

15. The neck-based personal attenuation rating (PAR) system according to claim 11 wherein the ambient noise level detector is carried by the computing device.

16. The neck-based personal attenuation rating (PAR) system according to claim 11 wherein the listener operates the audiometer via the response button on the touchscreen.

17. The neck-based personal attenuation rating (PAR) system according to claim 11 wherein the audiometer is configured to generate tones.

18. The neck-based personal attenuation rating (PAR) system according to claim 17 wherein the listener operates the audiometer by holding down the response button when the tone is heard, and releases the response button when the tone is no longer heard.

19. The neck-based personal attenuation rating (PAR) system according to claim 11 wherein the audiometer and the computing device are wirelessly coupled together.

20. The neck-based personal attenuation rating (PAR) system according to claim 11 wherein the headphones comprise spaced apart earcups, with the earcups being angled away from the listener when the headphones are placed around the neck of the listener.
